(19) United States
(12) Patent Application Publication
(10) Pub. No.: US 2014/0228915 A1
(43) Pub. Date: Aug. 14, 2014

(54) PAD FOR USE WITH A PHOTOTHERAPY SYSTEM

(71) Applicant: Steven Gardner, Columbia, SC (US)

(72) Inventor: Steven Gardner, Columbia, SC (US)

(21) Appl. No.: 13/766,260

(22) Filed: Feb. 13, 2013

(51) Int. Cl. A61N 5/06

(52) U.S. Cl. CPC A61N 5/0621
USPC 607/90; 29/592.1

(57) ABSTRACT

In an embodiment, a pad for use with a phototherapy system includes a first plastic layer having a surface facing the user in a central region. The first plastic layer defined by a peripheral edge has an average percent light transmission in a 440 nm to 480 nm band of at least 85%. A second plastic layer has the same structure and light transmission properties. The peripheral edges are connected. The first and second plastic layer central regions are spaced apart forming a closed cavity. The first and second plastic layer central regions are also connected forming a thermal bridge and dividing the cavity into a plurality of subcavities that fluidly communicate. The cavity includes a fluid medium having average percent light transmission of at least 85%. The combination of first and second plastic layers and fluid is sufficiently flexible to form a 2-T bend or tighter.

### TECHNICAL FIELD

This invention is directed generally to a pad for use with a phototherapy system.

### BACKGROUND

Phototherapy has long been used for treatment of certain ailments. An example of such ailments is jaundice, particularly in infants. Approximate 60% of infants born in the United States year become clinically jaundiced. Phototherapy for treating jaundice is commonly delivered using light emitting diodes (LEDs). The LED circuitry, in certain embodiments, is flexible or rigid and often covered in a rigid sheath.

The LED circuitry may be uncomfortable for an infant as the LED circuitry is relatively hard and initially cold, being at least 15° C. less than an infant's core body temperature. While certain embodiments of the LED circuitry may be sterilizable, the infant may have frequent and loose bowel movements necessitating time-consuming and costly cleaning and re-sterilization. What is needed is a component that cooperates with the LED circuitry that makes the infant comfortable while being treated by phototherapy, but which is very inexpensive to use and maintain.

### SUMMARY

In at least one embodiment, a pad for use with a phototherapy system having an LED phototherapy light source spaced apart from a user includes a first plastic layer having a surface facing the user in a central region. The first plastic layer has an average percent light transmission in a 440 nm to 480 nm band of at least 85%. The first plastic layer is defined by a peripheral edge. The pad also includes a second plastic layer having a central region that has an average light transmission in the 440 nm to 480 nm band of at least 85%. The second plastic layer is defined by a peripheral edge. The second layer peripheral edge is opposed to and connected to the first plastic layer peripheral edge on all peripheral edges. A first portion of the second plastic layer central region is spaced apart from a first portion of the first plastic layer central region forming a closed cavity. A second portion of the second plastic layer central region is connected to a second portion of the first plastic layer central region forming a thermal bridge and dividing the cavity into a plurality of subcavities. The subcavities fluidly communicate with each other. The closed cavity includes a fluid having average percent light transmission in the 440 nm to 480 nm band of at least 85%. The combination of the first plastic layer, the second plastic layer, and the fluid is sufficiently flexible to form a 2-T bend or tighter.

In another embodiment, a pad for use with a phototherapy system having an LED phototherapy light source spaced apart from a user includes a first plastic layer having a surface facing the user and a transparent central region. The first plastic layer has an average percent light transmission in a 400 nm to 550 nm band of at least 90%. The first plastic layer is defined by peripheral edge. The first plastic layer also includes a pocket portion of the first plastic layer that has a first disposition and a second disposition. The pad also includes a second plastic layer having a central region being defined by peripheral edge. A pocket portion of the second plastic layer has a first disposition and a second disposition. The second plastic layer peripheral edge is opposed to and connected to the first plastic layer peripheral edge on at least two peripheral edges. A first portion second plastic layer central region is spaced apart from a first portion of the first plastic layer central region forming a closed cavity. A second portion of the second plastic layer central region is connected to a second portion of the first plastic layer central region forming a thermal bridge. The thermal bridge divides the cavity into a plurality of subcavities. The closed cavity includes a fluid having average percent light transmission in the 400 nm to 550 nm band of at least 85%. The first and second plastic layer connections therebetween have sufficient strength to withstand increasing pressure of the fluid ranging from 2 times ambient pressure to 5 times ambient pressure during a sterilization time period.

In yet another embodiment, a method for use of a pad with a phototherapy system having an LED phototherapy light source spaced apart from the user and having a treatment area includes forming a sealed bag having a first plastic layer that has a light-transmitting central region and peripheral edges. The first plastic layer is adjacent to the user. The sealed bag has a second plastic layer having a central area and peripheral edges. The first and second plastic layer central regions are spaced apart to form a cavity. The method also includes selecting a light-transmitting fluid having average percent light transmission in a 400 nm to 550 nm band of at least 85%. The method also includes filling the cavity with the light-transmitting fluid to a thickness ranging from 1 cm to 3 cm. The cavity is sealed with a fluid-tight seal between the first and second plastic layer peripheral edges. The LED phototherapy light source is positioned adjacent to the second plastic layer and cooperates with the treatment area. The treatment area is treated with a light from the LED phototherapy light source. The combination of the sealed bag thickness, cavity thickness, and light-transmitting fluid average percent light transmission is adapted to provide 50% to 99% light transmission intensity reduction in the 400 nm to 550 nm band when comparing the initial light intensity of the LED phototherapy light source in the 400 nm to 550 nm band to a final intensity of the LED light at the user in the same frequency band.

### BRIEF DESCRIPTION OF THE DRAWINGS

FIG. 1 schematically illustrates an isometric view of a phototherapy system according to at least one embodiment;

FIG. 2 schematically illustrates a cross-sectional view of a phototherapy system along axis 2-2 in FIG. 1 according to at least one embodiment;

FIGS. 3A and 3B schematically illustrates a top view of a phototherapy system in a first condition and in a second condition according to at least one embodiment;

FIG. 4 schematically illustrates a cross-sectional view of a phototherapy system along axis 4-4 of FIG. 3B according to at least one embodiment;

FIG. 5 schematically illustrates a cross-sectional view of a phototherapy system according to at least one embodiment;

FIG. 6 schematically illustrates a top view of a phototherapy system according to at least one embodiment;

FIG. 7 schematically illustrates a cross-sectional view of a phototherapy system along axis 7-7 of FIG. 6 according to at least one embodiment; and

FIG. 8 diagrammatically illustrates a method of use of a phototherapy system according to at least one embodiment.

### DETAILED DESCRIPTION

Except where expressly indicated, all numerical quantities seen the descriptions in claims, indicated amounts of material or conditions of reaction and/or use are to be understood as modified by the word “about” in describing the broadest scope of the present invention. Practice within the numerical limits stated should be desired and independently embodied. Ranges of numerical limits may be independently selected from data provided in the tables and description. The description of the group or class of materials as suitable for the purpose in connection with the present invention implies that the mixtures of any two or more of the members of the group or classes are suitable. The description of constituents in chemical terms refers to the constituents at the time of addition to any combination specified in the description and does not necessarily preclude chemical interaction among constituents of the mixture once mixed. The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same techniques previously or later referenced for the same property. Also, unless expressly stated to the contrary, percentage, “parts of,” and ratio values are by weight, and the term “polymer” includes “oligomer,” “co-polymer,” “terpolymer,” “pre-polymer,” and the like.

It is also to be understood that the invention is not limited to specific embodiments and methods described below, as specific composite components and/or conditions to make, of course, vary. Furthermore, the terminology used herein is used only for the purpose of describing particular embodiments of the present invention and is not intended to be limiting in any way.

It must also be noted that, as used in the specification and the pending claims, the singular form “a,” “an,” and “the,” comprise plural reference unless the context clearly indicates otherwise. For example, the reference to a component in the singular is intended to comprise a plurality of components.

Throughout this application, where publications are referenced, the disclosure of these publications in their entirety are hereby incorporated by reference into this application to more fully describe the state-of-art to which the invention pertains.

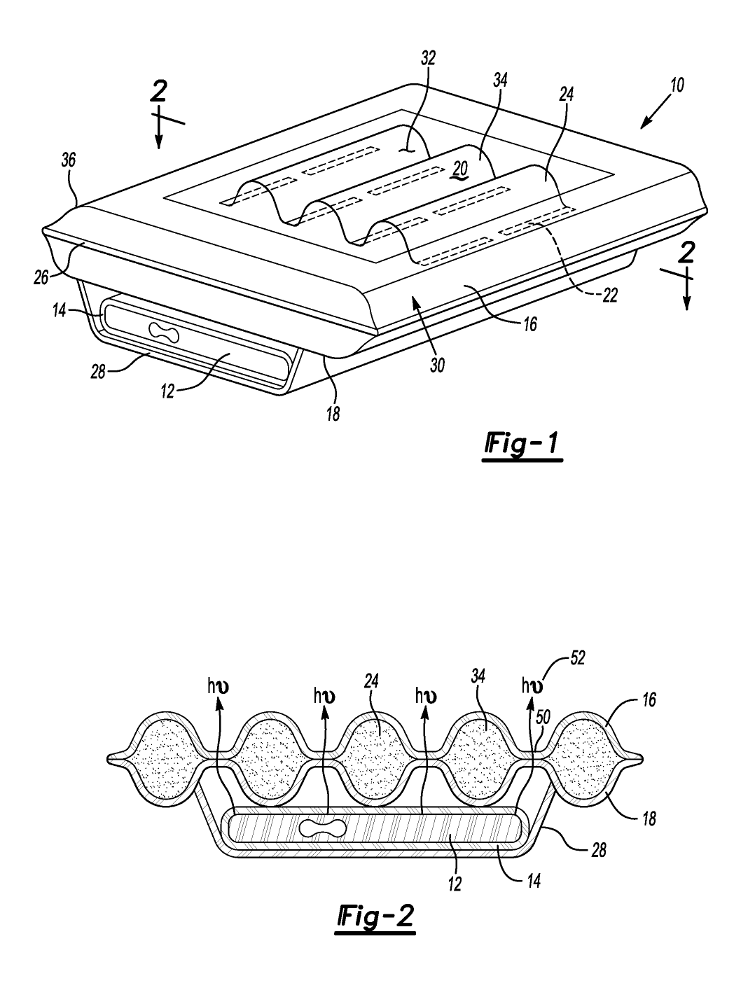

Turning now to FIG. 1, a phototherapy system 10 is schematically illustrated, in an isometric view according to at least one embodiment. Phototherapy system 10 includes an LED phototherapy light source 12 encased in an optimal sheath 14. Particularly advantageous LED phototherapy light sources 12 are disclosed in U.S. published application numbers 2006/0100675, 2007/0208397, 2010/0106228, which are all incorporated in their entireties by reference. A pad 30 is positioned between LED phototherapy light source 12 and a user (not shown). Pad 30 includes a first plastic layer 16 and a second plastic layer 18 which are spaced apart in a central light-transmitting region 32 to form cavity 20. It is understood that the first plastic layer 16 and the second plastic layer 18 may be either from a unitized sheet or separate sheets. A thermal break 22 separates cavity 20 into a plurality of subcavities 24. In at least one embodiment, a portion of subcavities 24 fluidly communicate between themselves. Cavity 20 is filled with a fluid medium 34. Fluid medium 34, in certain embodiments, may be distributed uniformly or non-uniformly within certain subcavities 24. First plastic layer 16 and second plastic layer 18 each have a peripheral edge 26. Peripheral edges 26 of first plastic layer 16 and second plastic layer 18 are connected forming a fluid-tight seal 36. In certain embodiments, a third plastic layer 28 is connected to first plastic layer 16 or the second plastic layer 18 and encompasses LED phototherapy light source 12 and sheath 14 holding LED phototherapy light source 12 and sheath 14 adjacent to second plastic layer 18 in order to minimize the distance that LED phototherapy light source 12 photons must travel to reach a treatment area on the user.

Turning now to FIG. 2, phototherapy system 12 is schematically illustrated in a cross-sectional view according to at least one embodiment. First plastic layer 16 connects to second plastic layer 18 at a thermal bridge 50. In subcavities 24, fluid medium 34 provides insulation between the LED phototherapy light source 12 which, in general, produces heat when operational. LED phototherapy light source 12 emits photons 52 passing through central light-transmitting region 32 of pad 30.

First plastic layer 16, in at least one embodiment, in central light-transmitting region 32 is transparent to the wavelengths emitted by LED phototherapy light source 12. In another embodiment, first plastic layer 16 in central light-transmitting region 32 is translucent to the wavelengths emitted by LED phototherapy light source 12. In yet another embodiment, first plastic layer 16 in central light-transmitting region 32 transmits at least 90% of the initial light from LED phototherapy light source 12 impinging on first plastic layer 16. In yet another embodiment, first plastic layer 16 in central light-transmitting region 32 transmits at least 85% of the initial light from LED phototherapy light source 12.

In at least one embodiment, first plastic layer 16 in central light-transmitting region 32 transmits at least 85% of light in the wavelengths ranging from 400 nm to 550 nm. In another embodiment, first plastic layer 16 in central light-transmitting region 32 transmits at least 85% of light in the wavelengths ranging from 440 nm to 480 nm. In another embodiment, first plastic layer 16 in central light-transmitting region 32 transmits at least 90% of light in wavelengths ranging from 400 nm to 550 nm. In yet another embodiment, first plastic layer 16 in central light-transmitting region 32 transmits at least 90% of the light in wavelengths ranging from 440 nm to 480 nm.

First plastic layer 16, in at least one embodiment, has a thickness ranging from 0.07 mm to 0.5 mm. In another embodiment first plastic layer 16 has a thickness ranging from 0.09 mm to 0.4 mm.

First plastic layer 16, in at least one embodiment, has at least one dimension greater than an LED phototherapy light source 12 dimension. In another embodiment, first plastic layer 16 has a minimum dimension ranging from 12 cm to 40 cm. In yet another embodiment, first plastic layer 16 has a maximum dimension ranging from 18 cm to 225 cm.

First plastic layer 16, in at least one embodiment, is capable of being sterilized without damage with 121° C. heat (i.e. have a melting point above 171° C.), ethylene oxide gas, and/or gamma-ray exposure. First plastic layer 16, in another embodiment, and has extremely low extractable monomers meeting levels dictated in Food and Drug Administration requirements.

First plastic layer 16, in at least one embodiment, is comprised of a thermoplastic polymer. Non-limiting examples of the thermoplastic polymer include relatively inexpensive commodity resins, polyvinyl chloride (PVC), ethylene vinyl acetate (EVA), polyamide resins, and polyolefins, including metallocene-catalyzed polyolefins. In at least one embodiment, the thermoplastic polymer is a flexible thermoplastic polymer. In at least one embodiment, the flexible thermoplastic polymer is more flexible than the LED phototherapy light source 12. In at least one embodiment, the flexible polymer has a flexural rigidity with a stiffness related to an elastic modulus ranging from 0.05 GN/m² to 3 GN/m². It is advantageous for the first plastic layer 16 to be relatively inexpensive to make economically feasible the option to dispose of pad 30 when treatment is completed avoiding the costs of cleaning and sterilization and/or exposure to bodily fluids, such as a baby's feces typically resulting from phototherapy treatment to reduce the baby's bilirubin quantity. Pad 30 can therefore be disposable. The pad 30 can be made of a biodegradable material to ease in disposal. Alternatively, a transparent cover or sheet can surround the upper surface of the pad (or the entire pad) in which the sheet is disposable after treatment is completed.

In at least one embodiment, first plastic layer 16 includes an oriented polymer. In another embodiment, first plastic layer 16 includes a biaxially-oriented polymer. In yet another embodiment, first plastic layer 16 includes an isotropic polymer.

Second plastic layer 18, in at least one embodiment, in central light-transmitting region 32 is transparent to the wavelengths emitted by LED phototherapy light source 12. In another embodiment, second plastic layer 18 in central light-transmitting region 32 is translucent to the wavelengths emitted by LED phototherapy light source 12. In yet another embodiment, second plastic layer 18 in central light-transmitting region 32 transmits at least 90% of the initial light from LED phototherapy light source 12 impinging on first plastic layer 16. In yet another embodiment, second plastic layer 18 in central light-transmitting region 32 transmits at least 85% of the initial light from LED phototherapy light source 12.

In at least one embodiment, second plastic layer 18 in central light-transmitting region 32 transmits at least 85% of light in the wavelengths ranging from 400 nm to 550 nm. In another embodiment, second plastic layer 18 in central light-transmitting region 32 transmits at least 85% of light in the wavelengths ranging from 440 nm to 480 nm. In another embodiment, second plastic layer 18 in central light-transmitting region 32 transmits at least 90% of light in wavelengths ranging from 400 nm to 550 nm. In yet another embodiment, second plastic layer 18 in central light-transmitting region 32 transmits at least 90% of the light in wavelengths ranging from 440 nm to 480 nm. In yet another embodiment, first plastic layer 16 transmits more light than second plastic layer 18 in central light-transmitting region 32. Is advantageous for first plastic layer 16 and second plastic layer 18 to transmit as much light as possible from LED phototherapy light source 12 because that allows the phototherapy system 10 to use either less intense or fewer bulbs to generate the photons 52 necessary for the treatment as well as to extend the operational life of phototherapy system 10 between recharges of the system or replacement of the batteries in LED phototherapy light source 12.

Second plastic layer 18, in at least one embodiment, has a thickness ranging from 0.07 mm to 0.5 mm. In another embodiment second plastic layer 18 has a thickness ranging from 0.09 mm to 0.4 mm. In yet another embodiment, second plastic layer 18 has a different thickness than first plastic layer 16. In yet another embodiment, second plastic layer 18 is thicker than first plastic layer 16. It is advantageous to minimize the thickness of first plastic layer 16 so as to maximize the amount of photons 52 transmitting through first plastic layer 16. It is also advantageous to have second plastic layer 18 thicker to support LED phototherapy light source 12 as well as to provide sufficient mass for bonding third plastic layer 28 to second plastic layer 18. It is additionally advantageous to have a thicker second plastic layer 18 in order to define a shape of pad 30 suitable for supporting the treatment area of the user.

Second plastic layer 18, in at least one embodiment, has at least one dimension greater than a LED phototherapy light source 12 dimension. In another embodiment, second plastic layer 18 has a minimum dimension ranging from 12 cm to 40 cm. In yet another embodiment, second plastic layer 18 has a maximum dimension ranging from 18 cm to 225 cm. in yet another embodiment, second plastic layer 18 is larger than first plastic layer 16.

Second plastic layer 18, in at least one embodiment, is capable of being sterilized without damage with 121° C. heat (i.e. have a melting point above 171° C.), ethylene oxide gas, and/or gamma-ray exposure. Second plastic layer 18, in another embodiment, and has extremely low extractable monomers meeting levels dictated in Food and Drug Administration requirements.

Second plastic layer 18, in at least one embodiment, is comprised of a thermoplastic polymer. Non-limiting examples of the thermoplastic polymer include polyvinyl chloride (PVC), ethylene vinyl acetate (EVA), polyamide resins, and polyolefins, including metallocene-catalyzed polyolefins. In at least one embodiment, the thermoplastic polymer is a flexible thermoplastic polymer. In at least one embodiment, the flexible thermoplastic polymer is more flexible than the LED phototherapy light source 12. In at least one embodiment, the flexible polymer has a flexural rigidity with a stiffness related to an elastic modulus ranging from 0.05 GN/m² to 3 GN/m². In at least one embodiment, the flexible polymer in second plastic layer 18 is stiffer than the flexible polymer in first plastic layer 16. It is advantageous for the second plastic layer 18 to be relatively inexpensive to make economically feasible the option to dispose of pad 30 when treatment is completed avoiding the costs of cleaning and sterilization or exposing technicians to bodily fluids or debris, such as a baby's feces typically resulting from phototherapy treatment to reduce the baby's bilirubin quantity. Sheath 14, first plastic layer 16, second plastic layer 18, and pad can be made of a biodegradable material.

In at least one embodiment, second plastic layer 18 includes an oriented polymer. In another embodiment, second plastic layer 18 includes a biaxially-oriented polymer. In yet another embodiment, second plastic layer 18 includes an isotropic polymer.

In at least one embodiment, pad 30 includes thermal breaks 50 present in an area up to 25% of the area of pad 30. In another embodiment, pad 30 includes thermal breaks 50 present in an area ranging from 5% to 20% of the area of pad 30. It is advantageous for pad 30 to have a minimum amount of thermal breaks in order to provide the user with effective insulation from heat emitted from the LED phototherapy light source 12. It is also advantageous for having sufficient thermal breaks 50 in combination with first plastic layer 16 tensile strength and second plastic layer 18 tensile strength to resist expansion pressures when pad 30 is sterilized by heat causing, in at least embodiment, increasing pressure ranging from 2 times to 5 times the initial pressure.

In at least one embodiment, fluid medium 34 in central light-transmitting region 32 transmits at least 85% of light in the wavelengths ranging from 400 nm to 550 nm. In another embodiment, fluid medium 34 in central light-transmitting region 32 transmits at least 85% of light in the wavelengths ranging from 440 nm to 480 nm. In another embodiment, fluid medium 34 in central light-transmitting region 32 transmits at least 90% of light in wavelengths ranging from 400 nm to 550 nm. In yet another embodiment, fluid medium 34 in central light-transmitting region 32 transmits at least 90% of the light in wavelengths ranging from 440 nm to 480 nm. It is advantageous for fluid medium 34 to transmit as much as light as is possible in the wavelengths of the treatment because fluid medium 34 is the thickest portion of pad 30.

Fluid medium 34, in at least one embodiment, has a thickness ranging from 1 cm to 3 cm. In another embodiment, fluid medium 34 has a thickness ranging from 1.5 to 2.5 mm.

Fluid medium 34, in at least one embodiment is comprised of a gas, a liquid, and/or a solid. Non-limiting examples of fluid medium 34 include dry air, moist air, warm air, cool air, water, glycol, a gel, and transparent particles of metal oxides.

In at least one embodiment, fluid medium 34 flows within cavity 20 or subcavities 24. In another embodiment, fluid medium 34 is static within cavity 20 or subcavities 24. In yet another embodiment, fluid medium 34 vaporizes or condenses to adapt to the amount of heat that needs to be added or withdrawn from pad 30 during a treatment. It is also advantageous for fluid medium 34 to be relatively inexpensive to make economically feasible the option dispose of pad 30 when treatment is completed avoiding the costs of cleaning and sterilization or exposing technicians to bodily fluids or debris. In certain embodiments, fluid medium 34 is a liquid or a liquid and corresponding gas in equilibrium. It is advantageous to have liquid that takes up more heat of vaporization before converting to a gas, which limits the increase in pressure within cavity 20 and/or subcavities 24. The limited increase in pressure permits thinning the first plastic layer 16 thickness and/or second plastic layer 18 thickness because pad 30 no longer has to resist as great pressure changes resulting for expansion of a gas-only fluid medium during temperature increases associated with thermal sterilization. Reducing the plastic thickness reduces the weight of pad 30 having an advantageous effect of making it easier to carry pad 30, and, in certain embodiments, including the treatment recipient, such as when the baby undergoes bilirubin reduction treatment. Prolonged touching the baby early in life, such as made possible by weight reduction of phototherapy pad 10 during bilirubin reduction treatment, has been reported as developmentally important for the baby.

In at least one embodiment, the combination of first plastic layer 16, second plastic layer 18, and fluid medium 34 has a thickness ranging 0.2 cm to 4 cm. In another embodiment, the combination of first plastic layer 16, second plastic layer 18, and fluid medium 34 has a thickness ranging 0.5 cm to 2 cm.

In at least one embodiment, pad 30 is bendable to cooperate with the desirable flexibility of LED phototherapy light source 12. In certain embodiments, the LED phototherapy light source 12 flexibility ranges from deformability sufficient to follow a human form, to a rigid panel that does not deform under 10 lbf/in². In at least one embodiment, pad 30 may be folded on itself in a 2-T bend or tighter. The test method includes bending the pad 30 about a mandrel through 180 degrees without the pad 30 showing cracking in the surface as evident under 10-power magnification. The first mandrel involves a thin sheet. This is a 0-T (i.e. 0-thickness mandrel) bend. The second mandrel is the thickness of pad 30. This is a 1-T bend. The third mandrel has twice the thickness of pad 30, and comprises a 2-T bend. For example, a 1-T bend is tighter than a 2-T bend. It should be obvious that incremental limits of thickness such as a 1.5-T bend, may be tested without exceeding the scope or spirit of the embodiments. In certain embodiments, it is desirable that pad 30 be able to be sufficiently flexible to render a 2-T bend or tighter. In another embodiment, pad 30 is sufficiently flexible to render a 1-T bend or tighter. In yet another embodiment, pad 30 is sufficiently flexible to render a 0-T bend. Increasing flexibility is desirable to accommodate flexibility of LED phototherapy light source 12 as well as providing a cushioning surface for the user, such as a baby, and allowing the light from LED phototherapy light source 12 to expose at least 8% of the user during treatment according to at least one embodiment. According to another embodiment, flexibility of pad 30 relative to LED phototherapy light source 12 allows the light from LED phototherapy light source 12 to expose at least 13% of the user, which represents the typical area of a user's back, during treatment. According to another embodiment, flexibility of pad 30 relative to LED phototherapy light source 12 allows the light from LED phototherapy light source 12 to expose at least 25% of the user during treatment advantageously speeding the completion of treatment.

In at least one embodiment, central light-transmitting region 32 of pad 30 has a total light reduction in a range of 50% to 99% of initial intensity LED phototherapy light source 12 relative to the final intensity of LED phototherapy light reaching the user. In another embodiment, central light-transmitting region 32 of pad 30 has a total light reduction in a range of 70% to 97% of initial intensity LED phototherapy light source 12 relative to the final intensity of LED phototherapy light reaching the user. In at yet another embodiment, central light-transmitting region 32 of pad 30 has a total light reduction in a range of 80% to 95% of initial intensity LED phototherapy light source 12 relative to the final intensity of LED phototherapy light reaching the user. In at least one embodiment, the light reduction occurs in wavelengths in a band from 400 nm to 550 nm. In another embodiment, the light reduction occurs in wavelengths in a band from 440 nm to 480 nm.

In at least one embodiment, pad 30 allows delivery of an average of patient treatment units (PTUs) ranging from 80 PTUs to 250 PTUs measured at the user. In another embodiment, pad 30 allows delivery of the average of PTUs ranging from 100 PTUs to 200 PTUs measured at the user. In another embodiment, pad 30 allows delivery of the average of PTUs ranging from 110 PTUs to 150 PTUs measured at the user. PTUs are calculated by the light intensity in microwatts times the area of the user exposed to the phototherapy treatment. It is advantageous to maximize the average PTUs per treatment to speed the completion of phototherapy. But, the average PTUs per treatment must be restricted such that undesirable damage to the user's skin or other organs does not occur because of excessive phototherapy light exposure.

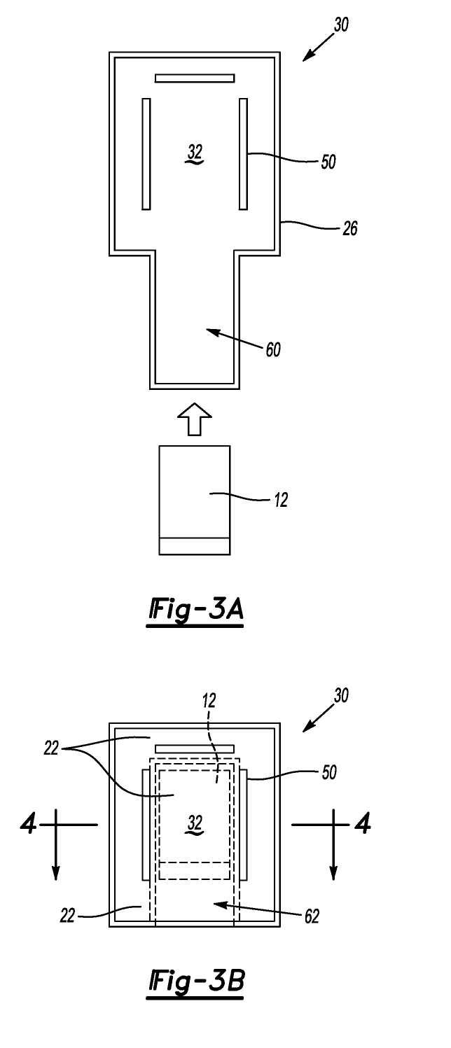

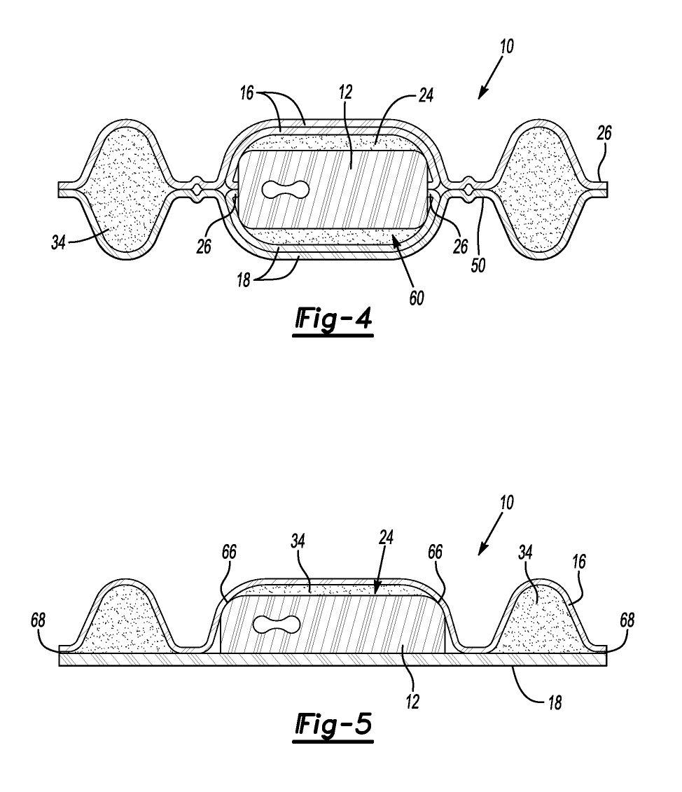

Turning now to FIGS. 3A, 3B, and 4, an embodiment is schematically illustrated in a top view in a first condition, a top view in a second condition, and a cross-sectional view along axis 4-4 of FIG. 3B. In at least one embodiment, the first condition includes first plastic layer 16 and second plastic layer 18 bonded together at peripheral edge 26. The shape of pad 30 includes a protrusion 60 capable of being inverted upon itself to form a polygonal intrusion, such as a pocket 62, when the first condition is changed to the second condition. In at least one embodiment, protrusion 60 is a polygonal shape. In another embodiment, protrusion 60 is a rectangular shape. Into pocket 62 LED phototherapy light source 12 may be inserted in certain embodiments. Protrusion 60, in at least one embodiment, exceeds the dimensional size of LED phototherapy light source 12. When pad 30 includes pocket 62, in general, there is subcavity 24 defined by at least one of first plastic layer 16 or second plastic layer 18 and LED phototherapy light source 12. In at least one embodiment, one or more subcavities 24 fluidly communicate with others subcavities 24. In at least one embodiment, one or more subcavities 24 are sealed off from others subcavities 24. In at least one embodiment, one or more subcavities 24 include fluid medium 34. Placing LED phototherapy light source 12 into pocket 62 advantageously places LED phototherapy light source 12 closer to the user thereby speeding the phototherapy treatment or reducing the energy necessary to power LED phototherapy light source 12 because the distance to the user is shortened while still providing desired cushioning for the user when a rigid LED phototherapy light source 12 is used. Using pad 30 with protrusion 60/pocket 62 advantageously reduces the expense of additional thermal welding of a third layer, such as third plastic layer 28, to second plastic layer 18. Thermal welding of thin thicknesses of plastic layers often results in burning small holes in plastic layers releasing fluid medium 34. It is also understood that while two portions of first plastic layer 16, or two portions of second plastic layer 18, are illustrated as being adjacent one another, fluid medium 34 may also be disposed between the two portions of either layer as a result of trapping fluid medium 34 between the portions of either layer when protrusion 60 is inverted upon itself when forming pocket 62.

Turning now to FIG. 5, phototherapy system 10 is schematically illustrated in a cross-sectional view according to at least one embodiment. In at least one embodiment first plastic layer 16 is thinner than second plastic layer 18. First plastic layer 16 includes an elastomeric polymer it can be stretched as LED phototherapy light source 12 is inserted. Where LED phototherapy light source 12 interferes with first plastic layer 16, a tight seal 66 is formed defining one or more subcavities 24 defined by first plastic layer 16 and LED phototherapy light source 12. In such subcavities 24, fluid medium 34 may be present to provide cushioning between LED phototherapy light source 12 and the user. Each plastic layer may be formed by most conventional plastic shaping processes. Non-limiting examples of shaping processes include sheet extrusion and thermoforming.

In at least one embodiment, first plastic layer 16 is connected to second plastic layer 18 by a layer 68. Non-limiting examples of layer 68 include an adhesive and a double-sided tape. It should be understood that any relatively fluid-tight seal-forming composition may be used in layer 68 without exceeding the scope or spirit of the embodiment. In at least one embodiment, first plastic layer 16 and second plastic layer 18 are bonded using a hot stamping method.

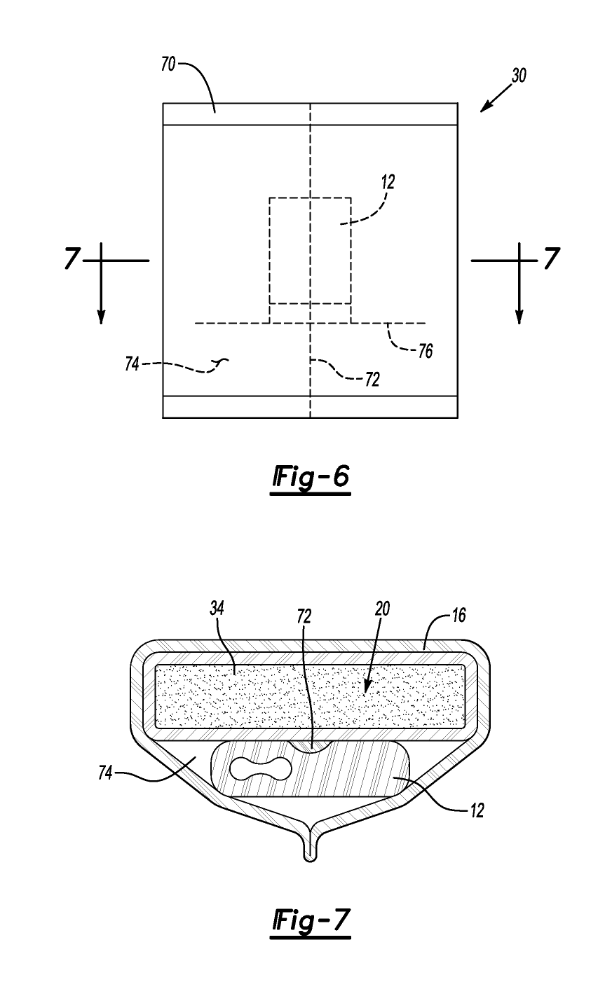

Turning now to FIGS. 6 and 7, photo therapy system 10 is schematically illustrated in a top view and a cross-sectional view along axis 7-7 according to at least one embodiment. Pad 30 may be formed from a single sheet of plastic using a flow wrapping machine. Pad 30 may include form, fill, and wrap structure where a flap 70 is formed at each end of a tube formed by wrapping a single sheet of plastic around a longitudinal axis. The sides of the single sheet of plastic are bonded together with a fluid-tight seal, such as a fin 72, forming cavity 20. Fluid medium 34 may be captured in cavity 20 during the flow wrapping process. It is understood that multiple serial flow wrapping processes may be combined so as to form an optional second cavity 74 that may be opened to insert LED phototherapy light source 12 into second cavity 74.

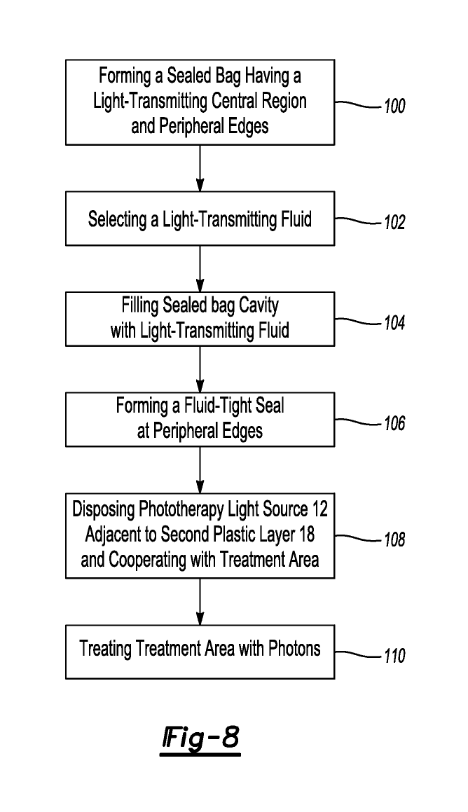

Turning now to FIG. 8, a method for use of pad 30 is diagrammatically illustrated. In step 100, a sealed bag is formed having a first plastic layer that has a light-transmitting central region and peripheral edges. The first plastic layer is adjacent to the user and being adjacent to a second plastic layer having a central area peripheral edges. The first and second plastic layer peripheral edges are capable of being connected to form a fluid-tight seal therebetween. The first and second plastic layer central regions are spaced apart to forming cavity. In step 102, a light-transmitting fluid having average percent light transmission in a 400 nm to 550 nm band that is at least 85%. In step 104 cavities filled with the light-transmitting fluid. The cavity has a thickness when the light-transmitting fluid is present in the range of from 1 cm to 3 cm. In step 106, a fluid-tight seal is formed at the first and second plastic layer peripheral edges. In step 108, the LED phototherapy light source 12 is positioned adjacent to second plastic layer 18 and cooperates with the treatment area. The treatment area is treated with a light from LED phototherapy light source 12 in step 110. A combination of the sealed bag thickness the cavity thickness in the light-transmitting fluid average percent light transmission is adapted to provide a percent light transmission in the 400 nm to 550 nm band that has an intensity reduction percentage in the range of 50% to 99% of an initial intensity of the LED phototherapy light in the 400 mm to 550 nm band at the phototherapy light source to a final intensity of the LED phototherapy light source 12 in the 400 nm 500 nm band at the user.

In any embodiment, first plastic layer 16, second plastic layer 18, or any part of pad 30 can be made of a material that includes small individual “impact softeners,” such as a bubble pack or BUBBLEWRAP®. An example of such material is Model No. S-12846, sold by ULINE®.

It should be understood that while the figures show first plastic layer 16, second plastic layer 18 and third plastic layer 28 are shown as sealed or connected around the peripheral of the pad 30, the plastic layers 16, 18, 28 can be connected or otherwise attached at only two or more peripheral edges. In this fashion, a pouch can be formed by attaching some, but not all, of the peripheral edges.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A pad for use with a phototherapy system having an LED phototherapy light source separated from a user, the pad comprising:
a first plastic layer having a surface facing the user and a central region, the first plastic layer having an average percent light transmission in a 440 nm to 480 nm band of at least 85%, the first plastic layer being defined by a peripheral edge; and
a second plastic layer having a central region having an average percent light transmission in the 440 nm to 480 nm band of at least 85% and being defined by a peripheral edge and the second plastic layer peripheral edge being opposed to and connected to the first plastic layer peripheral edge at all peripheral edges, a first portion of the second plastic layer central region being spaced apart from a first portion of the first plastic layer central region forming a closed cavity, a second portion of the second plastic layer central region being connected to a second portion of the first plastic layer central region forming a thermal bridge and dividing the cavity into a plurality of subcavities, the subcavities fluidly communicating with each other, wherein the closed cavity is filled with a fluid medium and has an average percent light transmission in the 440 nm to 480 nm band of at least 85%, wherein the combination of the first plastic layer, the second plastic layer, and the fluid medium is sufficiently flexible to form a 2-T bend or tighter.

2. The pad of claim 1, further comprising:
a third layer having one or more pieces which are connected to the second plastic layer for removably attaching the LED phototherapy light source to the pad.

3. The pad of claim 1, wherein the combination of the first plastic layer, the second plastic layer, and the fluid medium have a thickness ranging from 0.2 cm to 4 cm.

4. The pad of claim 1, wherein the combination of the first plastic layer thickness and percent light transmission in the 440 nm to 480 nm band, the second plastic layer thickness and percent light transmission in the 440 nm to 480 nm band, and the fluid thickness and percent light transmission in the 440 nm to 480 nm band have an intensity reduction percentage in a range of 50% to 99% of an initial intensity of the LED phototherapy light source in the 440 nm to 480 nm band at the phototherapy light source to a final intensity of the LED phototherapy light source in the 440 nm to 480 nm band at the user.

5. The pad of claim 1, wherein the pad has a surface area greater than that the of phototherapy light source.

6. The pad of claim 5, wherein the pad has an average of patient treatment units (PTUs) ranging from 100 PTUs to 200 PTUs at the user when exposed to photons from the LED photothereapy light source adjacent to the second plastic layer.

7. The pad of claim 1, wherein the thermal bridge area is present at up to 25% of the pad area.

8. The pad of claim 1, wherein the first plastic layer thickness ranges from 0.07 mm to 0.5 mm.

9. The pad of claim 1, wherein the first plastic layer has a melting point of at least 171° C.

10. The pad of claim 1, wherein the second plastic layer thickness is different from the first plastic layer thickness.

11. The pad of claim 1, further comprising a transparent outer layer that is removable such that the transparent outer layer is disposable and replaceable after treatments.

12. The pad of claim 1, wherein the second plastic layer stiffness is greater than the first plastic layer stiffness.

13. The pad of claim 1, wherein the pad is disposable.

14. A pad for use with a phototherapy system having an LED phototherapy light source separated from a user, the pad comprising:
a first plastic layer having a surface facing the user and a transparent central region, the first plastic layer having an average percent light transmission in a 400 nm to 550 nm band of at least 90%, the first plastic layer being defined by a peripheral edge, a pocket portion the first plastic layer having a first disposition and a second disposition; and
a second plastic layer having a central region being defined by a peripheral edge, a pocket portion of the second plastic layer having a first disposition and a second disposition, the second plastic layer peripheral edge opposed to and connected to the first plastic layer peripheral edge on at least two peripheral edges, a first portion of the second plastic layer central region being spaced apart from a first portion of the first plastic layer central region forming a closed cavity filled with a fluid medium, a second portion of the second plastic layer central region being connected to a second portion of the first plastic layer central region forming a thermal bridge and dividing the cavity into a plurality of subcavities, the closed cavity including a fluid having an average percent light transmission in the 400 nm to 550 nm band of at least 85%, wherein the first and second plastic layer and connections therebetween have sufficient strength to withstand an increase in pressure of the fluid medium ranging from 2 times ambient pressure to 5 times ambient pressure during a sterilization time period.

15. The pad of claim 14, wherein the first plastic layer and the second plastic layer pocket portions in the first disposition form a polygonal protrusion from the pad.

16. The pad of claim 14, wherein the first plastic layer and the second plastic layer pocket portions in the second disposition form a polygonal intrusion into the cavity adapted to retain the LED phototherapy light source.

17. The pad of claim 14, wherein the fluid medium comprises a warming medium.

18. A method of use of a pad with a phototherapy system having an LED phototherapy light source separated from a user and having a treatment area, the method comprising the steps of:
forming a sealed bag having a first plastic layer having a light-transmitting central region and peripheral edges, the first plastic layer being adjacent to the user and a second plastic layer having a central area and peripheral edges, the first and second plastic layer central regions being spaced apart to form a cavity;
selecting a light-transmitting fluid medium having an average percent light transmission in a 400 nm to 550 nm band of at least 85%;
filling the cavity with the light-transmitting fluid medium, the cavity having a thickness ranging from 1 cm to 3 cm;
forming a fluid-tight seal between the first and second plastic layer peripheral edges;
positioning the LED phototherapy light source adjacent to the second plastic layer and cooperating with the treatment area; and
treating the treatment area with a light from the LED phototherapy light source, wherein a combination of the sealed bag thickness, the cavity thickness and light-transmitting fluid medium average percent light transmission being adapted to provide a percent light transmission in the 400 nm to 550 nm band have an intensity reduction percentage in a range of 50% to 99% of an initial intensity of the LED phototherapy light source in the 400 nm to 550 nm band at the phototherapy light source to a final intensity of the LED phototherapy light source in the 400 nm to 550 nm band at the user.

19. The method of claim 18, wherein forming the sealed bag includes a hot stamping process.

20. The method of claim 18, further comprising the step of connecting the first plastic layer and the second plastic layer disposed with the cavity forming a plurality of subcavities.
